SEQUENCE LISTING (2 sequences)

SEQ ID NO: 1 - DNA, 1074 bp, Phanerochaete chrysosporium
  CDS (1)..(1074)
gcagtctgtccagacggcacccgcgtcactaacgcagcttgttgcgcatttattccactggctcaagacctccaggagaccctcttccagggcgactgcggtgaagatgcgcatgaggtcattcgtcttaccttccacgacgccattgctatctctcagagcctgggaccccaggccggcggtggtgctgacggctccatgctgcacttcccgaccatcgagccgaatttttcggcgaacaacggcattgacgactccgtgaacaaccttatccccttcatgcagaagcacaacacgatcagcgccgctgacctcgtccagttcgcgggagccgttgctctcagtaactgccccggcgcccctcgcctcgagttcctcgctggtcgcccgaacacgactattcccgcagtcgagggcctcatccctgagccgcaggacagtgtcaccaaaattctacaacgcttcgaggacgcagggaacttctcgccttttgaggtcgtatccctcctcgcctctcacactgttgctcgtgcagacaaggtcgacgagaccatcgacgccgcacccttcgattccacgcctttcactttcgacacccaggtcttcctcgaggtccttctgaagggtaccggcttccctggatcgaacaacaacaccggtgaggtcatgtccccacttcccctcggcagcggcagcgacacgggcgagatgcgcctgcagtctgactttgcgctcgcgcgcgacgagcgcacggcgtgcttctggcagtcgttcgtcaacgagcaggagttcatggcggcgagcttcaaggccgcgatggcgaagctcgcgatcctcggccacagccgcagcagcctcatcgactgcagcgacgtcgtccccgtgccgaagcccgccgtcaacaagcccgcgacgttccccgcgacgaagggccccaaggacctcgacacactcacgtgcaaggccctcaagttcccgacgctgacctctgaccccggtgctaccgagaccctcatcccccactgctccaacggcggcatgtcctgccctggtgttcagttcgatggccctgcc

SEQ ID NO: 2 - protein, 358 aa, Phanerochaete chrysosporium
AVCPDGTRVTNAACCAFIPLAQDLQETLFQGDCGEDAHEVIRLTFHDAIAISQSLGPQAGGGADGSMLHFPTIEPNFSANNGIDDSVNNLIPFMQKHNTISAADLVQFAGAVALSNCPGAPRLEFLAGRPNTTIPAVEGLIPEPQDSVTKILQRFEDAGNFSPFEVVSLLASHTVARADKVDETIDAAPFDSTPFTFDTQVFLEVLLKGTGFPGSNNNTGEVMSPLPLGSGSDTGEMRLQSDFALARDERTACFWQSFVNEQEFMAASFKAAMAKLAILGHSRSSLIDCSDVVPVPKPAVNKPATFPATKGPKDLDTLTCKALKFPTLTSDPGATETLIPHCSNGGMSCPGVQFDGPA

(19) United States
(12) Patent Application Publication
(10) Pub. No.: US 2003/0166184 A1
(43) Pub. Date: Sep. 4, 2003

(54) Process for stabilizing oxidase, stabilized oxidase, mutant oxidase and DNA

(75) Inventors: Haruo Takahashi, Aichi (JP); Chie Imamura, Aichi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kabushiki Kaisha Toyota Chuo Kenkyusho, Aichi-gun (JP)

(21) Appl. No.: 09/870,825

(22) Filed: Jun. 1, 2001

(30) Foreign Application Priority Data
Jun. 2, 2000 (JP) 2000-170443
Nov. 9, 2000 (JP) 2000-341859
Mar. 9, 2001 (JP) 2001-066097

(51) Int. Cl. C12N 9/00

(52) U.S. Cl. 435/183

(57) ABSTRACT

The surface of oxidase is stabilized by stabilizing the inner structure responsible for the enzyme function of the oxidase via the alteration of unstable amino acids therein into stable amino acids, and/or immobilizing the oxidase in a structure unit with structural stability and of a given inner diameter. The inner structure of oxidase is stabilized by altering specific unstable amino acids positioned within a 12-angstrom radius range from the active center of the oxidase or altering specific unstable amino acids facing the hydrogen peroxide-binding pocket of the oxidase. The process enhances the stability of the oxidase, particularly the stability of oxidase with lignin degradation activity against hydrogen peroxide.

### BACKGROUND OF THE INVENTION

1. Field of the Invention

The present invention relates to a process for stabilizing oxidase and a stabilized oxidase. The invention also relates to a mutant oxidase, and DNA encoding the mutant oxidase.

2. Description of the Related Art

Researches have been carried out in recent years for advantageously altering the function and stability of an enzyme or the substrate specificity thereof by designing the enzyme in a protein engineering manner.

Oxidases such as manganese peroxidase and lignin peroxidase have industrially significant uses in a wide range of fields. For example, such oxidases are used for degrading lignin as a coloring material in the process of producing paper pulp. As described in Japanese Patent Laid-Open No. 296949/1994, such oxidases are also used for plastic degradation. Thus, it is strongly demanded to provide an excellent mutant oxidase, in particular.

As to researches of mutant oxidase, a report has been made on the addition of veratolyl alcohol-binding site to lignin peroxidase (J. Exp. Med., 184, 831-837, 1996). There is also a report on the alteration of the substrate specificity of peroxidase (Japanese Patent Laid-Open No. 155570/1999).

Enzyme immobilization is effective for the improvement of enzyme stability. Conventionally, a method comprising directly immobilizing enzyme on resin beads and the like has been proposed. A microcapsule method has also been proposed, comprising coating enzyme with a translucent polymer film. A surface modification method has also been proposed, comprising modifying enzyme surface with polyethylene glycol or glycolipid. Furthermore, a sol-gel method for immobilizing enzyme has been proposed, comprising utilizing a sol or colloid suspension of acrylamide, polyvinyl alcohol, tetramethoxysilane (TMOS), silane with organic groups and the like to utilize the gelation reaction thereof (Kawakami et. al., J. Ferment. Bioeng., 84, 240-242, 1998).

Because an oxidase, namely peroxidase utilizes hydrogen peroxide for the enzyme reaction, hydrogen peroxide resistance is particularly significant. Despite great usefulness, lignin peroxidase and manganese peroxidase as oxidases of a type exerting lignin degradation activity disadvantageously have low hydrogen peroxide resistance. The problem is noticeable, for example, during enzymatic degradation of lignin using lignin peroxidase and manganese peroxidase in the production step of paper pulp. In other words, since these enzymes are inactivated unless hydrogen peroxide as an activating substance is controlled at a very low concentration, the efficiency of the enzymatic reaction can never be improved through the elevation of the concentration of hydrogen peroxide.

The background art described above relates to the alteration of oxidase, but never pays any attention to the stability or thermal stability of oxidase in the presence of hydrogen peroxide. U.S. Pat. No. 5,817,495 discloses an example where hydrogen peroxide resistance of an oxidase is enhanced via the alteration of the oxidase. However, the oxidase is not of a type exerting lignin degradation activity. Further, the patent merely shows the alteration of the oxidase via shuffling, and does not clearly describes any technical concept relating to the alteration of oxidase.

The background art relating to enzyme immobilization has been poor in terms of the resulting enzyme stability. The reason lies in that the enzyme is immobilized in its exposed state onto the surface of the carrier for immobilization and that the structural stability of the structural unit for such immobilization is insufficient. The sol-gel method of Kawakami, et al., can bring about a certain, but not sufficient, effect on enzyme stability.

In the background art, no researches have been conducted to study the effect of simultaneous alternation and immobilization of oxidase on the enzyme stability.

### SUMMARY OF THE INVENTION

It is an object of the present invention to remarkably improve the stability of oxidase, particularly hydrogen peroxide resistance of an industrially useful oxidase of a type exerting lignin degradation activity.

The present inventors have realized a mutant oxidase with excellent hydrogen peroxide resistance, through alteration, namely stabilization of the active center or hydrogen peroxide-binding pocket of an oxidase. Further, the inventors have succeeded in dramatically improving the hydrogen peroxide resistance by immobilizing such mutant in a structural unit with structural stability.

In a first aspect, the invention provides a process for stabilizing oxidase, comprising: an inner structure stabilization step of stabilizing an inner structure responsible for oxidase function; and a surface stabilization step of stabilizing oxidase surface.

According to the first aspect, oxidase can be stabilized from the inside by the inner structure stabilization step and can simultaneously be stabilized from the outside by the surface stabilization step. Consequently, it was confirmed that an enzyme stabilization effect far above the effect generally expected could be achieved for at least oxidase. Herein, the term “the effect generally expected” means generally expected synergistic effect, on the basis of the effect brought about when the inner structure stabilization step is singly practiced and the effect brought about when the surface stabilization step is singly practiced.

In a second aspect of the invention, the inner structure stabilization step according to the first aspect comprises the following step 1) and /or 2).

1) A step of altering at least one amino acid oxidizable or at least one amino acid with a steric configuration readily modifiable, among amino acids positioned within a radius range of 12 angstroms from the active center of oxidase, into a non-oxidizable amino acid or an amino acid with a steric configuration hardly modifiable.

2) A step of altering at least one amino acid oxidizable or at least one amino acid with a steric configurations readily modifiable among amino acids facing the hydrogen peroxide-binding pocket of oxidase, into a non-oxidizable amino acid or an amino acid with a steric configuration hardly modifiable.

The inner structure stabilization step described above in 1) in the second aspect serves to stabilize the active center of oxidase, as an important inner structure. The inner structure stabilization step described above in 2) in the second aspect serves to stabilize the active center of oxidase, as an important inner structure. Herein, the detailed mechanism of the inner structure stabilization step described above in 1) and 2) to dramatically stabilize oxidase has not yet been elucidated, specifically as to how the inner structure stabilization step can cooperate with the surface stabilization step described below.

In a third aspect of the invention, the surface stabilization step immobilizes the enzyme in the structure unit of an inner diameter 1.2 times or more the diameter of the enzyme and with structural stability.

In the third aspect, the surface of oxidase is firmly immobilized in the structure unit. Therefore, severe change of the steric configuration of oxidase is avoided, so that oxidase can be stabilized. Further, a certain inner space can be kept between the surface of oxidase and the structure unit. Therefore, a slight change of the steric configuration required for the expression of the oxidase activity is permissible, while the oxidase can be kept in contact with the substrate.

In a fourth aspect of the invention, the surface stabilization step comprises immobilizing oxidase in a structural unit of an inner diameter 1.2 times or more the diameter of the enzyme and with structural stability and forming the reticulated structure of a gelated substance in the opening of the structural unit and/or the inner space thereof.

In accordance with the fourth aspect, a large change in the steric configuration of the immobilized enzyme is more effectively avoided due to the reticulated structure of the gelated substance. Hence, the oxidase is sufficiently firmly immobilized and stabilized even in a relatively large structure unit. Because the reticulated structure of the gelated substance involves many channels or spaces, the required change of the steric configuration of the enzyme is permissible, which is essential for the expression of the enzyme activity. Since the opening or inner space of the structural unit is not completely occluded, the contact of the enzyme and the substrate is never inhibited.

In a fifth aspect, the invention provides a stabilized oxidase, which is prepared by practicing a process for stabilizing oxidase, according to any of the first to fourth aspects of the invention.

The stabilized oxidase in the fifth aspect can exert various actions and effects in accordance with the first to fourth aspects of the invention.

In a sixth aspect, the invention provides the stabilized oxidase, which is peroxidase with lignin degradation activity.

The stabilized oxidase in accordance with the sixth aspect is very useful in producing paper pulp and in other fields. Because such peroxidase has very low hydrogen peroxide resistance in nature, the advantage of the stabilization thereof is prominently great.

In a seventh aspect, the invention provides a stabilized oxidase having the residual activity of 100% in the presence of 10 mM hydrogen peroxide.

Such stabilized oxidase in accordance with the seventh aspect is particularly preferable for the purpose of enhancing the efficiency of the enzyme reaction by increasing the concentration of hydrogen peroxide to a higher level.

In an eighth aspect, the invention provides a mutant oxidase, which is prepared by partially or wholly altering oxidizable amino acids or amino acids with steric configurations readily modifiable, as positioned within a radius range of 12 angstroms from the active center of oxidase, into non- oxidizable amino acids or amino acids with steric configurations hardly modifiable.

It is believed that oxidase from native origin is inactivated due to the oxidation or alteration of oxidizable amino acids or amino acids with steric configurations readily modifiable by hydrogen peroxide in the proximity of the active center. In the mutant oxidase of the eighth aspect, the stability of the oxidase has been enhanced by modifying unstable amino acids in the proximity of the active center of oxidase from native origin into stable amino acids. Particularly, the stability thereof against hydrogen peroxide can be enhanced. In some case, all of oxidizable amino acids or amino acids with steric configurations readily modifiable in the proximity of the active center should be altered, while in other case, they should partially be altered, satisfactorily. Generally, similar alteration of oxidizable amino acids or amino acids with steric configurations readily modifiable, as positioned apart from the active center or other important inner structures, does not cause enzyme inactivation.

In a ninth aspect of the invention, the residual activity of the mutant oxidase in accordance with the eighth aspect in the presence of 1 mM hydrogen peroxide at 25° C., 5 minutes later, is 50% or more. Because the residual activity of oxidase from native origin under the same conditions decreases to about 10 to 20%, the mutant oxidase of the ninth aspect is very effective industrially.

In a tenth aspect of the invention, the mutant oxidase of the eighth aspect has a substitution of at least one methionine of the methionine molecules at positions 67, 237 and 273 in the oxidase of the amino acid sequence of SQ ID NO. 1 with leucine, isoleucine, valine, alanine, glycine or serine.

The mutant oxidase of the tenth aspect is shown as a representative example of the mutant oxidase of the eighth aspect.

In an eleventh aspect of the invention, a mutant oxidase is prepared by altering at least one oxidizable amino acid or amino acid with a steric configuration readily modifiable, among amino acids facing the hydrogen peroxide-binding pocket of oxidase, into a non-oxidizable amino acid or amino acid with a steric configuration hardly modifiable. Herein, amino acids to give adverse effects on oxidase functions through such alteration are not selected for the alteration.

The term “hydrogen peroxide-binding pocket” means a hollow pocket-like structure, which should be considered as a separate concept from the active center of oxidase. The hydrogen peroxide-binding pocket has a function such that the binding of hydrogen peroxide with the pocket triggers the emergence of radicals via the oxidation of iron molecules in the active center. The present inventors consider that the iron molecules in the active center get hardly oxidizable when the spatial structure of the hydrogen peroxide-binding pocket is modified, so that the activity of oxidase decreases. The commonest cause of the modification of the spatial structure is the modification of the steric configuration structures of amino acids facing the hydrogen peroxide-binding pocket.

In accordance with the eleventh aspect, unstable amino acids facing the hydrogen peroxide-binding pocket are stabilized, so that the stability of the oxidase is enhanced. Consequently, the stability of the oxidase against heat, acids, alkalis and the like can be improved greatly.

In a twelfth aspect of the invention, the residual activity of the mutant oxidase in accordance with the eleventh aspect in the presence of 0.3 mM hydrogen peroxide at 25° C., 5 minutes later, is 90% or more. Such stability is very preferable in practical use of oxidase.

In a thirteenth aspect of the invention, the mutant oxidase of the eleventh aspect has a substitution of the asparagine at position 81 in the oxidase of the amino acid sequence of SQ ID NO. 1 with serine.

The mutant oxidase of the thirteenth aspect is shown as a representative example of the mutant oxidase of the eleventh aspect.

In a fourteenth aspect of the invention, the mutant oxidase in accordance with the eighth or eleventh aspect is a mutant of oxidase with lignin degradation activity. The oxidase with lignin degradation activity typically includes manganese peroxidase or lignin peroxidase.

The stabilized oxidase of the fourteenth aspect is very useful in producing paper pulp or in other fields. Further, such peroxidase has such low resistance against hydrogen peroxide in nature, that the advantage of the stabilization is particularly great.

In a fifteenth aspect of the invention, the oxidizable amino acids or amino acids with steric configurations readily modifiable in accordance with the eighth or eleventh aspect are asparagine, glutamine, tryptophan, cysteine or methionine, while the non-oxidizable amino acids or amino acids with steric configurations hardly modifiable are alanine, leucine, isoleucine, valine, alanine, glycine or serine.

The alteration of amino acids in accordance with the fifteenth aspect exerts a particularly high effect on the stabilization of the mutant oxidase.

In a sixteenth aspect of the invention, the oxidizable amino acids or the amino acids with steric configurations readily modifiable in accordance with the eighth or eleventh aspect have steric configurations similar to those of the non-oxidizable amino acids or the amino acids with steric configurations hardly modifiable.

In the sixteenth aspect, unexpected damages of enzyme functions or the like which may occur due to such amino acid alteration can effectively be avoided.

In a seventeenth aspect, the invention provides DNA comprising the following nucleotide sequence 3) or 4):

3) the nucleotide sequence encoding the amino acid sequence of the mutant oxidase of the tenth aspect; and

4) the nucleotide sequence encoding the amino acid sequence of the mutant oxidase of the thirteenth aspect.

The above and other advantages of the invention will become more apparent from the following description and the accompanying drawings.

### BRIEF DESCRIPTION OF THE DRAWINGS

FIG. 1 is a schematic view of the steric configuration of manganese peroxidase;

FIG. 2 is a graph showing enzyme stability;

FIG. 3 is a graph showing enzyme stability;

FIG. 4 is a graph showing enzyme stability; and

FIG. 5 is a graph showing enzyme stability;

### DETAILED DESCRIPTION OF THE INVENTION

[Oxidase]

The oxidase type as a target of the alteration in accordance with the invention is not limited. Examples of preferable oxidases include: peroxidase and catalase of which the activating substance is hydrogen peroxide; and laccase and heme oxidase of which the activating substance is oxygen. The oxidase of the invention also includes oxidases with a substitution of the amino acids therein, which are not amino acids positioned within a radius range of 12 angstroms from the active center and amino acids facing the hydrogen peroxide-binding pocket.

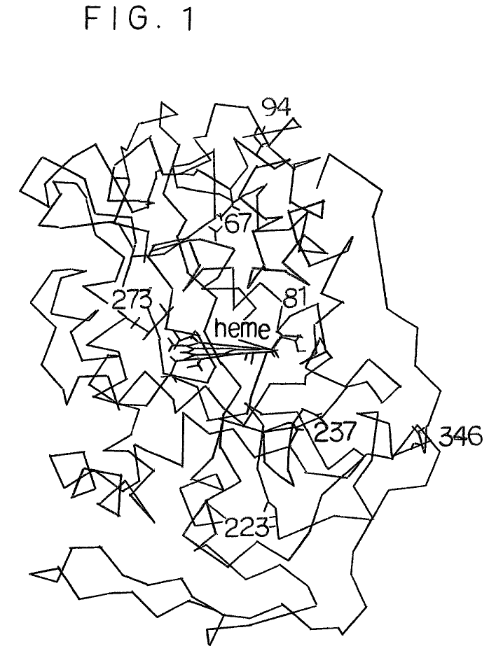

Particularly preferable oxidase is peroxidase with lignin degradation activity, particularly manganese peroxidase or lignin peroxidase. The amino acid sequence of native-type manganese peroxidase derived from white rod fungus as one of the specific examples thereof is shown as SQ ID No. 1 in the sequence listing, while the steric configuration of the active type is schematically shown in FIG. 1. Oxidases except for those described above are also subjects of the alteration. Depending on the difference in organisms as the origin, oxidases have variable amino acid sequences, steric configurations, active center structures, hydrogen peroxide-binding pocket structures, the types of amino acids facing the hydrogen peroxide-binding pocket and the like.

As the “oxidase”, in accordance with the invention, not only common oxidase protein molecules may be used, but also the active units thereof (enzyme fragments containing the active site) may also be used singly.

[Inner Structure Stabilization Step]

The inner structure stabilization step of the invention is not limited to specific contents, as long as the step can stabilize the inner structure responsible for the enzyme function of oxidase. A preferable example of the inner structure stabilization step comprises altering oxidizable amino acids or amino acids with readily modifiable steric configurations occupying given spatial positions in the steric configuration of oxidase, into non-oxidizable amino acids or amino acids with steric configurations hardly modifiable. Herein, even oxidizable amino acids or amino acids with steric configurations readily modifiable may potentially cause the inhibition of the enzyme function, through the alteration thereof into other amino acids. Such amino acids are exceptionally not preferable as subjects for the alteration.

Herein, preferable examples of the “given spatial positions” include: 1) positions within a radius range of 12 angstroms from the active center of oxidase; and 2) positions facing the hydrogen peroxide-binding pocket of oxidase. The term “facing hydrogen peroxide-binding pocket” means that a part or most of the structure of the amino acid is exposed to the hollow space of the hydrogen peroxide-binding pocket. One or both of the amino acids occupying the spatial positions 1) and 2) as described above may satisfactorily be subjects for the alteration.

[Amino acid alteration]

The amino acids at the given spatial positions and as subjects of the alteration (these are referred to as “altering amino acids” hereinafter) are oxidizable by acids, alkalis,heat and the like,or readily modifiable of their steric configurations. The altering amino acids are preferably altered into amino acids hardly oxidizable by acids, alkalis, heat and like,or into amino acids with steric configurations hardly modifiable (referred to as “altered amino acids” hereinafter). For the alteration of these amino acids, more preferably, the altering amino acids and the altered amino acids have similar steric configurations.

In view of the respects described above, preferable examples of the altering amino acids include sulfur-containing amino acids and amino acids with side chains having amino groups. More specifically, the altering amino acids include asparagine, glutamine, tryptophan, cysteine or methionine. As such cysteine, generally, cysteine molecules forming an S—S bond to contribute to the retention of the steric configuration of the protein are excluded from the subject of the alteration. Free cysteine not forming any S—S bond should be a subject for the alteration. It may sometimes be effective to simply delete the altering amino acids from the amino acid sequence of oxidase from native origin. However, the deletion may cause an unexpected adverse effect on the steric configuration of the resulting oxidase, and the like.

Preferable examples of the altered amino acids include aliphatic amino acids or amino acids with small side chains, more specifically including alanine, leucine, isoleucine, valine, serine or glycine.

In some case, the alteration of a single amino acid among the altering amino acids at the given spatial positions brings about a good stabilization effect, while, in some other case, the alteration of two or more or all of the altering amino acids at the given spatial positions brings about a good stabilization effect. The requirements vary, depending on the type of a specific oxidase, so the number of amino acids to be altered is not determined definitely. If the number of amino acids being positioned in the proximity of the active center of oxidase or facing the hydrogen peroxide- binding pocket as altering candidates is three, i.e. A, B and C, all of the three amino acids should sometimes be altered, while alteration of one or two of A, B and C may sometimes bring about definite effects.

In case that oxidase is native-type manganese peroxidase from white rod fungus, the altering amino acid positioned within a radius range of 12 angstroms from the active center of the oxidase is preferably at least one of the methionine molecules at positions 67, 237 and 273 in the amino acid sequence of SQ ID NO. 1. The altered amino acid in that case is preferably leucine, isoleucine, valine, alanine, glycine or serine, particularly preferably leucine.

Further, the altering amino acid facing the hydrogen peroxide-binding pocket in the native-type manganese peroxidase as an oxidase is preferably asparagine at position 81 in the amino acid sequence of SQ ID NO. 1. The altered amino acid in that case is particularly preferably serine. Alanine, leucine, valine or glycine is also preferable.

[Preferable Properties of Stabilized Oxidase or Mutant Oxidase]

It is expected for the stabilized oxidase or mutant oxidase of the invention that the enzyme activity at a given concentration of an activating substance can be relatively stabilized or that the enzyme activity in the presence of a given concentration of an activating substance can be thermally stabilized relatively.

A particularly preferable standard of the effects attained in accordance with the first or fifth aspect of the invention is the 100% residual activity in the presence of 10 mM hydrogen peroxide of the stabilized oxidase.

A particularly preferable standard of the effects attained in accordance with the eighth aspect is the 50% or higher residual activity of the mutant oxidase at 25° C. in the presence of 1 mM hydrogen peroxide, 5 minutes later.

A particularly preferable standard of the effects attained in accordance with the eleventh aspect is the 90% or higher residual activity of the mutant oxidase at 25° C. in the presence of 0.3 mM hydrogen peroxide, 5 minutes later.

[Protein, DNA, Vector, Recombinant Transformant and Method for Preparing Protein]

The amino acid sequence and nucleotide sequence of native-type manganese peroxidase derived from white rod fungus (Phanerochaete crysosporium) are shown in SQ ID NO. 1, while FIG. 1 schematically shows the steric configuration of the active type thereof.

A protein prepared by substituting at least one methionine of the methionine molecules at positions 67, 237 and 273 in the native-type manganese peroxidase with leucine, isoleucine, valine, alanine, glycine or serine is a potential typical example in accordance with the first, fifth or eighth aspect. Such mutant protein, but having the amino acid sequence where at least one amino acid other than these amino acids is substituted, deleted or added and exerting oxidase activity, may also be a potential typical example of the first, fifth or eighth aspect.

The protein prepared by substituting the asparagine at position 81 in the native-type manganese peroxidase with serine is a potential typical example in accordance with the first, fifth or eleventh aspect. Such mutant protein, but having the amino acid sequence where at least one amino acid other than these amino acids is substituted, deleted or added and exerting oxidase activity, may also be a potential typical example of the first, fifth or eleventh aspect.

The oxidase prepared by the inner structure stabilization step for alteration of various amino acids can be prepared by known appropriate methods. The oxidase can be prepared highly efficiently, for example, by constructing an expression vector carrying DNA encoding an intended mutant oxidase, integrating the expression vector in an appropriate host cell for culturing and recovering the generated mutant oxidase through an appropriate process. The DNA can be prepared by known appropriate methods, for example highly efficient amplification, comprising preparing a primer for the nucleotide sequence encoding a partial amino acid sequence containing an amino acid to be altered and effecting PCR, using the primer and the gene DNA of native-type peroxidase as template.

The type and details of the expression vector, the type of the host cell, the culture conditions of the host cell, and the separation and purification methods of the mutant thus generated may appropriately be determined as required.

[Surface stabilization step]

The surface stabilization step in accordance with the invention is not specifically limited of its details, as long as the step can stabilize the surface of oxidase. A preferable example of the surface stabilization step comprises immobilizing oxidase in a structure unit of an inner diameter of at least 1.2 times the enzyme diameter, preferably about 1.2 to 1.5 times the enzyme diameter and with structural stability. Another preferable example of the surface stabilization step comprises immobilizing oxidase in a structure unit of an inner diameter of about 5 to 10 nm and with structural stability. Further, more preferably, the reticulated structure of a gelated substance is formed in the opening and/or inner space of the structure unit.

Not only oxidase prepared by the inner structure stabilization step but also oxidase without the inner structure stabilization step can be immobilized in combination in the structure unit. Two or more types of oxidases prepared by the inner structure stabilization step can also be immobilized in combination. Oxidase and a wide variety of enzymes to be related therewith in a series of reaction can be immobilized in combination. In these cases, two or more types of the enzymes may be immobilized in separate structure units in the same porous material or the like, or may be immobilized in the same structure unit.

[Structure Unit and Porous Material]

As the structure unit, a material with structural stability and with the inner diameter described above is used. The structure unit may be composed of an inorganic material or an organic material such as organic polymer. Further, a structure unit with an anchor unit (element with functional groups conjugating the structure unit to the enzyme, such as hydroxyl group, amino group and silane derivatives) is particularly preferable in light of the strong enzyme stabilizing action. For the structure unit comprising an organic material, a polymer formation reaction is sometimes needed for coating the periphery of the enzyme through an anchor unit. The type of the polymer or the types of monomers composing the polymer are not specifically limited, unless they obstruct the purpose of the invention.

The structure unit comprising an inorganic material and having structural stability can be composed of for example various metal oxides such as silicic acid and alumina, and composite oxides of silicic acid with other metal oxides (inorganic porous materials). For forming a structure unit comprising silicic acid, for example, layered silicates such as Kanemite, alkoxysilane, silica gel, water glass and soda silicate can preferably be used.

For preparing the structure unit from an inorganic material, the inorganic material is mixed with a surfactant (template substance) for reaction, to form a surfactant/inorganic composite where the inorganic skeleton is formed around the micelle of the surfactant. Then, the surfactant is removed, for example through sintering at 400° C. to 600° C. or extraction in organic solvents, to form a mesopore of the same shape as that of the micelle of the surfactant in the inorganic skeleton (mesoporous silica material).

In case that silicon-containing compounds, for example silicic acid, is used as a starting material in a method for preparing the structure unit, a layered silicate such as Kanemite is first prepared. Then, the micelle is inserted in between the layers, while the layers with no presence of such micelle are conjugated together by means of the silicate molecule. Subsequently, the micelle is removed to form such pore. In another method, a silicon-containing material such as water glass is used as a starting material, the silicate monomer is accumulated around the micelle for polymerization to form silica, and the micelle molecule is removed therefrom to form the pore. In this case, the micelle is generally in a column shape, so that the resulting pore is in the shape of a column.

In these cases, the inner diameter of the structure unit can be regulated by modifying the length of the alkyl chain of the surfactant to modify the diameter of the micelle. Further, the addition of relatively hydrophobic molecules such as trimethylbenzene and tripropylbenzene in combination with the surfactant can swell the micelle, so that a structure unit of a larger inner diameter can be formed.

The forms of the structure unit include powder, granule, sheet, bulk, membrane and the like. The inner diameter of the structure unit, namely each pore, is preferably 1.2 times or more, more preferably about 1.2 to 1.5 times the diameter of the enzyme to be immobilized. Because the specific value of the inner diameter is determined depending on the enzyme diameter, the value can never be defined definitely. However, the value can be for example about 5 to 10 nm.

[Reticulated Structure of Gelated Substance]

The reticulated structure of the gelated substance to stabilize the oxidase immobilized in the structure unit is formed by the sol-gel method after the enzyme immobilization. In accordance with the invention, the term “sol-gel method” means a method for gelling a low-molecular raw material to constitute a high-molecular gelated substance in the co-presence of the immobilized enzyme in the sol or colloid suspension of the low-molecular raw material. The gelated substance formed by the sol-gel method is in a reticulated structure with many channels or spaces in the opening and/or inner space of the structure unit.

The type of the low-molecular raw material is not limited. For example, acrylamide, polyvinyl alcohol, tetramethoxysilane (TMOS) and silanes with organic groups as described above can be used. Generally, the raw materials composing the structure unit of the invention can be used effectively, including for example various metal oxides such as silicic acid and alumina, composite oxides of silicic acid with other types of metals, alkoxysilane, silica gel, water glass and silicate soda.

The conditions for carrying out the sol-gel method, for example the use of buffer solution, the concentration of a low-molecular raw material to be gelated in the sol or colloid suspension, pH adjustment, the requirement of heating or cooling during reaction and the like are not definitely limited. In case that alkoxysilane is used as such low-molecular raw material, more preferable conditions for carrying out the sol-gel method include the addition of dimethyl-dimethoxysilane (DMDMOS). The conditions also may include the addition of DMDMOS and tetramethoxysilane (TMOS) at a ratio of 1:1 to 1:3. The conditions further include the elevation of the pH during such gelation to 5 to 8.

### DESCRIPTION OF PREFERRED EMBODIMENTS

### EXAMPLES OF THE FIRST AND FIFTH ASPECTS

(Preparation of Mutant Manganese Peroxidase)

Manganese peroxidase from a native origin, namely native-type manganese peroxide from white rod fungus, having the active-type steric configuration shown in FIG. 1 and the amino acid sequence of SQ ID NO. 1, was the subject for alteration. Three methionine molecules at positions 67, 237 and 273 in the proximity of the heme region as the active center of the enzyme, namely in the 12-angstrom radius range from the enzyme active center, were modified into leucine to prepare a mutant manganese peroxidase [1]. In FIG. 1, these methionine molecules are individually marked with the corresponding numerical figures, 67, 237 and 273. The process of preparing the mutant followed the genetic engineering technique described above. Escherichia coli was used as the host cell.

The mutant protein generated as an inclusion body in Escherichia coli was dissolved in 8 M urea and purified on a His-tag purification column. Further, 5 μM hemin was added to the resulting purified product, which was then dialyzed against Tris buffer containing 1 mM calcium, to prepare the mutant manganese peroxide of the active type [altered MnP].

(Stability Test Against Hydrogen Peroxide)

Using the altered MnP, the residual activity thereof in hydrogen peroxide solutions at various concentrations was monitored and tested.

For assaying the activity, hydrogen peroxide was added to 25 mM succinate buffer ( pH 4.5) containing 0.5 mM manganese sulfate, 2 mM oxalic acid and 0.5 mM ABTS (2,2′-azinobis3-ethylbennzothiazoline-6-sulfonate), to prepare solutions at final various hydrogen peroxide concentrations. To the individual solutions were added 5 units of each of the altered MnP and manganese peroxide of native type [native-type MnP], for reaction at 25° C. for 5 minutes. Subsequently, the absorbance was measured at 405 nm to calculate the residual activity.

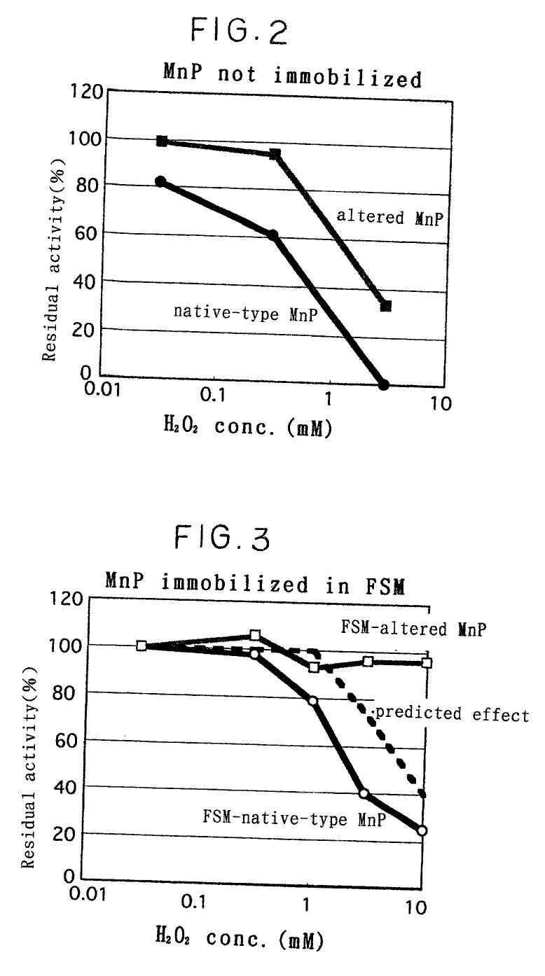

The results are shown in FIG. 2. As apparently shown in FIG. 2, the residual activity of the altered MnP as one example of the invention is far higher than that of the native-type MnP, in the presence of various concentrations of hydrogen peroxide. For example, the native-type MnP can readily lose the activity at the increase of the hydrogen peroxide concentration. However, the residual activity of the altered MnP in the presence of 1 mM hydrogen peroxide at 25° C. was 50% or more, 5 minutes later.

The altered MnP (1500 units) and the native-type MnP (1500 units) were added to 30 mg of a mesoporous silica porous material of a mean pore size of about 70 angstroms. Then, the resulting mixture was agitated overnight at 4° C., to recover the altered MnP immobilized on the mesoporous silica material [FSM-altered MnP] and the native-type MnP immobilized on the mesoporous silica material [FSM-native-type MnP].

These FSM-altered MnP and FSM-native-type MnP were left to stand in the presence of 1 to 10 mM hydrogen peroxide at 37° C. for one hour. Subsequently, the resulting MnPs were diluted to final 0.1 mM hydrogen peroxide concentration, to assay the residual activities thereof. The results are shown in FIG. 3. As apparently shown in the comparison between FIGS. 3 and 2, the stability of the FSM-altered MnP against hydrogen peroxide is increased than that of the altered MnP, and the stability of the FSM-native type MnP against hydrogen peroxide is increased than that of the native-type MnP.

It should be noted that the effect on the increase of the stability of the FSM-altered MnP over the stability of the altered MnP is far higher than the effect generally predicted (expressed in the graph with dotted line in FIG. 3) on the basis of the effect on the increase of the stability of the FSM-native-type MnP over the stability of the native-type MnP. For example, the residual activity of the FSM-altered MnP was retained at 100% when the MnP was left to stand in the presence of 10 mM hydrogen peroxide, while the residual activity of the FSM-native-type MnP was decreased to about 20% under the same conditions.

### EXAMPLE OF THE EIGHTH ASPECT

(Preparation of Mutant Manganese Peroxide)

In the same manner as in the “Examples of the first and fifth aspects”, a mutant of active-type manganese peroxidase [1] was recovered. By the same process, then, the three methionine molecules at positions 94, 223 and 346 in the outer periphery of the steric configuration of the native-type manganese peroxidase were modified into leucine, to prepare a mutant manganese peroxidase [2]. The active type thereof was also recovered. In FIG. 1, these methionine molecules are marked with the corresponding numerical figures of 94, 223 and 346.

(Stability Test Against Hydrogen Peroxide)

Using the mutant manganese peroxidase [1] and mutant manganese peroxidase [2], the residual activities thereof in hydrogen peroxide solutions at various concentrations were monitored and tested.

For assaying the activity, hydrogen peroxide was added to 25 mM succinate buffer (pH 4.5) containing 0.5 mM manganese sulfate, 2 mM oxalic acid and 0.5 mM ABTS, to prepare solutions at final various hydrogen peroxide concentrations. To the individual solutions were added 5 units of each of the altered mutants [1] and [2] and the manganese peroxide of native type, for reaction at 25° C. for 5 minutes. Subsequently, the absorbance was measured at 405 nm to calculate the residual activity.

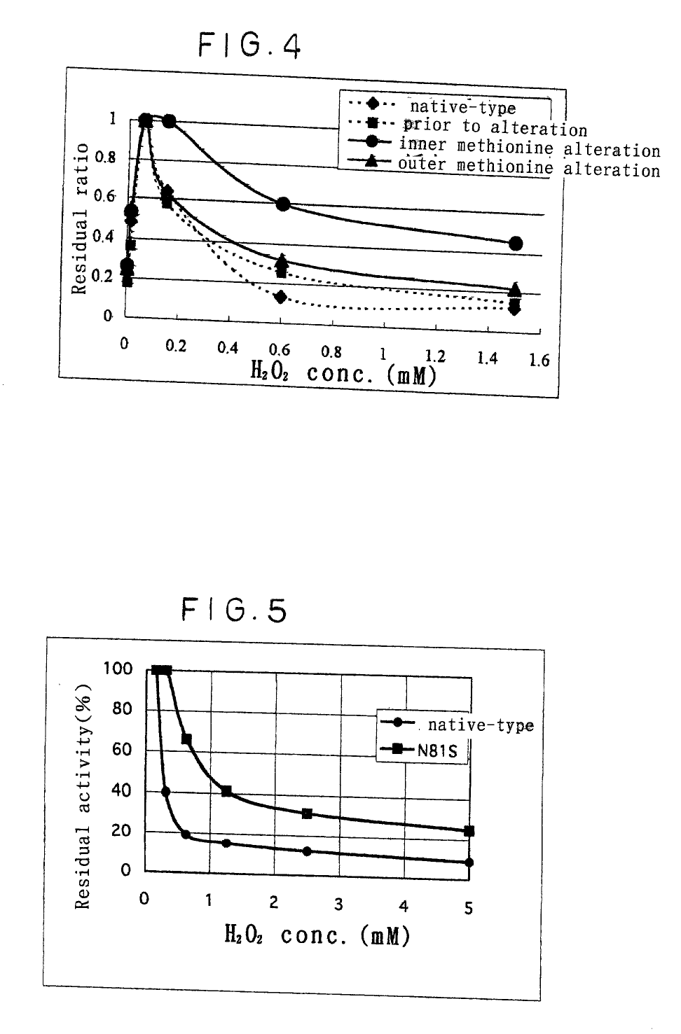

The results are shown in FIG. 4. In FIG. 4, the mark with “inner methionine alteration” represents mutant manganese peroxidase [1]. The mark with “outer methionine alteration” represents mutant manganese peroxidase [2]. The mark with “native type” represents native-type manganese peroxidase purified from white rod fungus. The mark with “prior to alteration” represents recombinant manganese peroxidase with no alteration.

As is apparent from FIG. 4, the residual activity of the mutant manganese peroxidase [1] as one example of the invention is far higher than that of the “native type”, “prior to alteration” or “outer methionine alteration”. In “inner methionine alteration”, the residual activity in the presence of 1 mM hydrogen peroxide at 25° C. was 50% or more, 5 minutes later. However, the residual activities of the “native type” and “prior to alteration” were 20% or less, while the residual activity of the “outer methionine alteration ” was below 30%.

### EXAMPLE OF THE ELEVENTH ASPECT

(Preparation of Mutant Manganese Peroxidase)

By altering asparagine as the amino acid at position 81 facing the hydrogen peroxide-binding pocket in the native-type manganese peroxidase of the amino acid sequence of SQ ID No. 1 into serine, a mutant manganese peroxidase was prepared. The asparagine was marked with the numerical FIG. 81 in FIG. 1. The process of preparing the mutant followed the same genetic engineering technique as described above. As the host cell, Escherichia coli was used.

The mutant protein generated as an inclusion body in Escherichia coli was dissolved in 8 M urea and purified on a His-tag purification column. Further, 5 μM hemin was added to the resulting purified product, which was then dialyzed against Tris buffer containing 1 mM calcium, to prepare the mutant manganese peroxide of the active type.

(Stability Test Against Hydrogen Peroxide)

Using the altered MnP, the residual activity thereof in hydrogen peroxide solutions at various concentrations was monitored and tested.

For assaying the activity, hydrogen peroxide was added to 25 mM succinate buffer (pH 4.5) containing 0.5 mM manganese sulfate, 2 mM oxalic acid and 0.5 mM ABTS, to prepare solutions at final various hydrogen peroxide concentrations. To the individual solutions were added 5 units of each of the mutant manganese peroxidase and the native-type manganese peroxidase purified from white rod fungus, for reaction at 25° C. for 5 minutes. Subsequently, the absorbance was measured at 405 nm to calculate the residual activity.

The results are shown in FIG. 5. In FIG. 5, the mark with “N81S” shows mutant manganese peroxidase. The mark with “native type” shows the native-type manganese peroxidase purified from white rod fungus.

As is apparent from FIG. 5, the residual activity of the mutant manganese peroxidase as one example of the invention is far higher than that of the native-type manganese peroxidase, in the presence of various concentrations of hydrogen peroxide. At a hydrogen peroxide concentration below 1 mM, the residual activity of the “native type” already decreases below 20%, which is substantially close to the inactivated state. In the presence of 1 mM hydrogen peroxide, the residual activity of “N81S” at 25° C. is at a value around 50%, 5 minutes later. Even in the presence of hydrogen peroxide at such a high concentration as 3 to 5 mM, further, the residual activity of “N81S” at 25° C. is around 30% or far above 20%, 5 minutes later.

While the preferred embodiments have been described, variations thereto will occur to those skilled in the art within the scope of the present inventive concepts which are delineated by the following claims.

What is claimed is:

1. A process for stabilizing oxidase, comprising
an inner structure stabilization step of stabilizing an inner structure responsible for oxidase function, and
a surface stabilization step of stabilizing oxidase surface.

2. The process for stabilizing oxidase according to claim 1, wherein the inner structure stabilization step comprises the following step 1) and/or 2):
1) altering at least one amino acid oxidizable or at least one amino acid with a steric configuration readily modifiable, among amino acids positioned within a radius range of 12 angstroms from the active center of oxidase, into a non-oxidizable amino acid or an amino acid with a steric configuration hardly modifiable; and
2) altering at least one amino acid oxidizable or at least one amino acid with a steric configuration readily modifiable among amino acids facing a hydrogen peroxide-binding pocket of oxidase, into a non-oxidizable amino acid or an amino acid with a steric configuration hardly modifiable.

3. The process for stabilizing oxidase according to claim 1, wherein the surface stabilization step immobilizes the enzyme in a structure unit of an inner diameter 1.2 times or more the diameter of the enzyme and with structural stability.

4. The process for stabilizing oxidase according to claim 1, wherein the surface stabilization step comprises immobilizing the oxidase in a structure unit of an inner diameter of 1.2 times or more the diameter of the enzyme and with structural stability and forming the reticulated structure of a gelated substance in the opening of the structural unit and/or the inner space thereof.

5. A stabilized oxidase, which is prepared by practicing a process for stabilizing oxidase according to claim 1.

6. The stabilized oxidase according to claim 5, wherein the stabilized oxidase is peroxidase with lignin degradation activity.

7. The stabilized oxidase according to claim 5, wherein the residual activity of the stabilized oxidase in the presence of 10 mM hydrogen peroxide is 100%.

8. A mutant oxidase, which is prepared by partially or wholly altering oxidizable amino acids or amino acids with steric configurations readily modifiable, as positioned within a radius range of 12 angstroms from the active center in the active-type steric configuration of oxidase, into non-oxidizable amino acids or amino acids with steric configurations hardly modifiable.

9. The mutant oxidase according to claim 8, wherein the residual activity of the mutant oxidase in the presence of 1 mM hydrogen peroxide at 25° C., 5 minutes later is 50% or more.

10. The mutant oxidase according to claim 8, wherein the mutant oxidase has a substitution of at least one methionine of the methionine molecules at positions 67, 237 and 273 in the oxidase of the amino acid sequence of SQ ID NO. 1 with leucine, isoleucine, valine, alanine, glycine or serine.

11. A mutant oxidase, which is prepared by altering at least one oxidizable amino acid or amino acid with a steric configuration readily modifiable among amino acids facing a hydrogen peroxide-binding pocket of oxidase, into at least one non-oxidizable amino acid or amino acid with a steric configuration hardly modifiable.

12. The mutant oxidase according to claim 11, wherein the residual activity of the mutant oxidase in the presence of 0.3 mM hydrogen peroxide at 25° C., 5 minutes later is 90% or more.

13. The mutant oxidase according to claim 11, wherein the mutant oxidase has a substitution of the asparagine at position 81 in the oxidase of the amino acid sequence of SQ ID NO. 1 with serine.

14. The mutant oxidase according to claim 8 or 11, wherein the mutant oxidase is a mutant of oxidase with lignin degradation activity.

15. The mutant oxidase according to claim 8 or 11, wherein the oxidizable amino acids or amino acids with steric configurations readily modifiable are asparagine, glutamine, tryptophan, cysteine or methionine, while the non-oxidizable amino acids or amino acids with steric configurations hardly modifiable are alanine, leucine, isoleucine, valine, alanine, glycine or serine.

16. The mutant oxidase according to claim 8 or 11, wherein the oxidizable amino acids or the amino acids with steric configurations readily modifiable have steric configurations similar to those of the non-oxidizable amino acids or the amino acids with steric configurations hardly modifiable.

17. DNA comprising the following nucleotide sequence 3) or 4):
3) the nucleotide sequence encoding the amino acid sequence of a mutant oxidase according to claim 10; and
4) the nucleotide sequence encoding the amino acid sequence of a mutant oxidase according to claim 13.
